(19) United States
(12) Patent Application Publication
(10) Pub. No.: US 2019/0382852 A1
(43) Pub. Date: Dec. 19, 2019

(54) Mitochondrial DNA deletion between about residues 12317-16254 for use in the detection of cancer

(71) Applicant: MDNA Life Sciences, Inc., West Palm Beach, FL (US)

(72) Inventors: Ryan Parr, Thunder Bay (CA); Jennifer Creed, Broomfield, CO (US); Kerry Robinson, Thunder Bay (CA); Andrea Maggrah, Thunder Bay (CA); Katrina Maki, Porcupine (CA); Gabriel Dakubo, Thunder Bay (CA); Brian Reguly, Thunder Bay (CA); Andrew Harbottle, Tyne and Wear (GB); Jude Alexander, Petawawa (CA)

(73) Assignee: MDNA Life Sciences, Inc., West Palm Beach, FL (US)

(21) Appl. No.: 16/558,108

(22) Filed: Sep. 1, 2019

Related U.S. Application Data
(63) Continuation-in-part of application No. 15/947,192, filed on Apr. 6, 2018, now Pat. No. 10,400,290.
Application No. 15/947,192 is a continuation of application No. 15/188,604, filed on Jun. 21, 2016, now abandoned.
Application No. 15/188,604 is a continuation of application No. 14/489,119, filed on Sep. 17, 2014, now abandoned.
Application No. 14/489,119 is a continuation of application No. 13/745,204, filed on Jan. 18, 2013, now abandoned.
Application No. 13/745,204 is a continuation of application No. 12/742,032, filed on Aug. 25, 2010, now abandoned.
(60) Provisional application No. 61/002,637, filed on Nov. 9, 2007.

(51) Int. Cl. C12Q 1/6886

(52) U.S. Cl. CPC C12Q 1/6886; C12Q 2600/158; C12Q 2600/156

(57) ABSTRACT

Methods and kits for predicting, diagnosing and monitoring cancer, wherein the methods comprise quantifying mitochondrial DNA mutations in biological samples. The methods of the invention may also be effective in screening for new therapeutic agents and treatment regimens and may also be useful for monitoring the response of a subject to a preventative or therapeutic treatment.

### CROSS-REFERENCE TO RELATED APPLICATIONS

This application is a Continuation in Part of U.S. patent application Ser. No. 15/947,192, filed Apr. 6, 2018, which is a Continuation of U.S. patent application Ser. No. 15/188,604, filed Jun. 21, 2016, which is a Continuation of U.S. patent application Ser. No. 14/489,119, filed Sep. 17, 2014, which is a Continuation of U.S. patent application Ser. No. 13/745,204, filed Jan. 18, 2013, which is a Continuation of U.S. patent application Ser. No. 12/742,032, filed Aug. 25, 2010, which is a National Stage Entry of PCT/CA2008/001956, filed Nov. 10, 2008, which claims priority from U.S. Application No. 61/002,637, filed Nov. 9, 2007. The entire contents of each of the aforementioned applications are incorporated herein by reference as if set forth in their entirety.

### SEQUENCE LISTING

A computer readable text file, entitled “Sequence Listing (11871/00283).txt”, created on Aug. 30, 2019, with a file size of about 32 kb contains the sequence listing for this application and is hereby incorporated by reference in its entirety.

### FIELD OF THE INVENTION

The present invention pertains to the field of mitochondrial genomics. In particular it is related to the detection of human mitochondrial genome mutations and their utility as an indicators of cancer.

### BACKGROUND OF THE INVENTION

### Mitochondrial DNA as a Diagnostic Tool

Mitochondrial DNA (mtDNA) sequence dynamics are important diagnostic tools. Mutations in mtDNA are often preliminary indicators of developing disease, often associated with nuclear mutations, and act as biomarkers specifically related to: disease, such as but not limited to, tissue damage and cancer from smoking and exposure to second hand tobacco smoke (Lee et al., 1998; Wei, 1998); longevity, based on accumulation of mitochondrial genome mutations beginning around 20 years of age and increasing thereafter (von Wurmb, 1998); metastatic disease caused by mutation or exposure to carcinogens, mutagens, ultraviolet radiation (Birch-Machin, 2000); osteoarthritis; cardiovascular, Alzheimer, Parkinson disease (Shoffner et al., 1993; Sherratt et al., 1997; Zhang et al, 1998); age associated hearing loss (Seidman et al., 1997); optic nerve degeneration and cardiac dysrhythmia (Brown et al., 1997; Wallace et al., 1988); chronic progressive external exophthalmoplegia (Taniike et al., 1992); atherosclerosis (Bogliolo et al., 1999); papillary thyroid carcinomas and thyroid tumours (Yeh et al., 2000); as well as others (e.g. Naviaux, 1997; Chinnery and Turnbull, 1999).

Mutations at specific sites of the mitochondrial genome can be associated with certain diseases. For example, mutations at positions 4216, 4217 and 4917 are associated with Leber's Hereditary Optic Neuropathy (LHON) (Mitochondrial Research Society; Huoponen (2001); MitoMap). A mutation at 15452 was found in 5/5 patients to be associated with ubiquinol cytochrome c reductase (complex III) deficiency (Valnot et al. 1999).

Specifically, these mutations or alterations include point mutations (transitions, transversions), deletions (one base to thousands of bases), inversions, duplications, (one base to thousands of bases), recombinations and insertions (one base to thousands of bases). In addition, specific base pair alterations, deletions, or combinations thereof have been found to be associated with early onset of prostate, skin, and lung cancer, as well as aging (e.g. Polyak et al., 1998), premature aging, exposure to carcinogens (Lee et al., 1998), etc.

### Prostate Cancer

Prostate cancer is a frequently diagnosed solid tumour that most likely originates in the prostate epithelium (Huang et al. 1999). In 1997, nearly 10 million American men were screened for prostate specific antigen (PSA), the presence of which suggests prostate cancer (Woodwell, 1999). Indeed, this indicates an even higher number of men screened by an initial digital rectal exam (DRE). In the same year, 31 million men had a DRE (Woodwell, 1999). Moreover, the annual number of newly diagnosed cases of prostate cancer in the United States is estimated at 179,000 (Landis et al., 1999). It is the second most commonly diagnosed cancer and second leading cause of cancer mortality in Canadian men. In 1997 prostate cancer accounted for 19,800 of newly diagnosed cancers in Canadian men (28%) (National Cancer Institute of Canada). It is estimated that 30% to 40% of all men over the age of forty-nine (49) have some cancerous prostate cells, yet only 20% to 25% of these men have a clinically significant form of prostate cancer (SpringNet—CE Connection, internet, www.springnet.com/ce/j803a.htm). Prostate cancer exhibits a wide variety of histological behaviour involving both endogenous and exogenous factors, i.e. socio-economic situations, diet, geography, hormonal imbalance, family history and genetic constitution (Konishi et al. 1997; Hayward et al. 1998). Although certain mtDNA alterations have been previously associated with prostate cancer, the need exists for further markers for the detection of prostate cancer.

### Breast Cancer

Breast cancer is a cancer of the glandular breast tissue and is the fifth most common cause of cancer death. In 2005, breast cancer caused 502,000 deaths (7% of cancer deaths; almost 1% of all deaths) worldwide (World Health Organization Cancer Fact Sheet No. 297). Among women worldwide, breast cancer is the most common cancer and the most common cause of cancer death (World Health Organization Cancer Fact Sheet No. 297). Although certain mtDNA alterations have been previously associated with breast cancer, for example in Parrella et al. (Cancer Research: 61, 2001), the need exists for further markers for the detection of breast cancer.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention pertains to mitochondrial DNA mutations for use in the detection of cancer. In accordance with an aspect of the present invention, there is provided a method of detecting a cancer in an individual comprising:

a) obtaining a biological sample from the individual;

b) extracting mitochondrial DNA (mtDNA) from the sample;

c) quantifying the amount of mtDNA in the sample having a deletion in the mtDNA sequence between about residue 12317 and about residue 16254 of the human mtDNA genome; and

d) comparing the amount of mtDNA in the sample having the deletion to at least one known reference value.

In accordance with another aspect of the present invention, there is provided a method of monitoring an individual for the development of a cancer comprising:

a) obtaining a biological sample;

b) extracting mitochondrial DNA (mtDNA) from the sample;

c) quantifying the amount of mtDNA in the sample having a deletion in the mtDNA sequence between about residue 12317 and about residue 16254 of the human mtDNA genome; and

d) repeating steps a) to c) over a duration of time;

wherein an increasing level of the deletion over the duration of time is indicative of cancer.

In accordance with another aspect of the present invention, there is provided a method of detecting a cancer in an individual comprising:

a) obtaining a biological sample from the individual;

b) extracting mitochondrial DNA (mtDNA) from the sample;

c) quantifying the amount of mtDNA in the sample having a sequence corresponding to the sequence as set forth in SEQ ID NO: 1 or SEQ ID NO: 2; and

d) comparing the amount of mtDNA in the sample corresponding to SEQ ID NO: 1 or SEQ ID NO: 2 to at least one known reference value.

In accordance with another aspect of the present invention, there is provided a diagnostic kit for carrying out the method of the invention comprising:

(a) material for collecting one or more biological samples; and

(b) suitable primers and reagents for detecting the mtDNA deletion.

In one aspect, there is provided a method of detecting breast or prostate cancer, or a genetic predisposition to breast or prostate cancer, in a human subject, the cancer being characterized by an elevated amount of mitochondrial DNA (mtDNA) having a deletion of 3926 base pairs within a region of mtDNA between nucleotides 12317 and 16254, with respect to SEQ ID NO. 3, of the human mtDNA genome, the method comprising:

a) contacting mtDNA extracted from a biological sample from the subject with at least one binding agent that specifically binds to a sequence of mtDNA having a spliced region after removal of the deletion;

b) quantifying the amount of mtDNA having the deletion by quantifying the amount of mtDNA bound to the at least one binding agent; and,

c) detecting the cancer or the predisposition to cancer where the quantified amount of mtDNA having the deletion is elevated in relation to at least one known reference value.

In another aspect, there is provided a method of detecting breast or prostate cancer, or a genetic predisposition to breast or prostate cancer, in a human subject, the cancer being characterized by an elevated amount of mitochondrial DNA (mtDNA) having a deletion in the human mtDNA genome, the deletion having a nucleotide sequence as set forth in SEQ ID NO: 1 or SEQ ID NO: 2, the method comprising:

a) contacting mtDNA extracted a biological sample from the subject with at least one binding agent that specifically binds to: i) a sequence of mtDNA having a spliced region after removal of the deletion; or ii) to a sequence of mtDNA comprising the rejoining site of the deletion sequence of SEQ ID NO: 1 or SEQ ID NO: 2 after the deletion sequence has re-circularized;

b) quantifying the amount of mtDNA having the deletion by quantifying the amount of mtDNA bound to the at least one binding agent; and,

c) detecting the cancer or the predisposition to cancer where the quantified amount of mtDNA having the deletion is elevated in relation to at least one known reference value.

In one aspect, there is provided a method of detecting breast or prostate cancer, or a genetic predisposition to breast or prostate cancer, in a human subject, the cancer being characterized by an elevated amount of mitochondrial DNA (mtDNA) having a deletion of 3926 base pairs within a region of mtDNA between nucleotides 12317 and 16254, with respect to SEQ ID NO. 3, of the human mtDNA genome, the method comprising:

a) contacting mtDNA extracted from a biological sample from the subject with a pair of PCR primers that specifically bind to a sequence of mtDNA having a spliced region after removal of the deletion, wherein one primer of the pair of primers has the nucleotide sequence as set forth in SEQ ID NO: 10;

b) quantifying the amount of mtDNA having the deletion by quantifying the amount of mtDNA bound to the primers; and,

c) detecting said cancer or said predisposition to cancer where the quantified amount of mtDNA having the deletion is elevated in relation to at least one known reference value.

In another aspect there is provided a method of quantifying, in a biological sample from a human subject, the amount of mitochondrial DNA (mtDNA) having a deletion of 3926 base pairs within a region of mtDNA between nucleotides 12317 and 16254 of SEQ ID NO. 3 corresponding to the human mtDNA genome, the method comprising:

a) contacting the biological sample with a pair of PCR primers, wherein one primer of the pair of primers specifically binds to a region of the mtDNA having a spliced region after removal of the deletion; and,

b) amplifying and quantifying the amount of mtDNA having the deletion using real-time PCR.

In another aspect, there is provided a kit for detecting breast or prostate cancer, or a genetic predisposition to breast or prostate cancer, in a human subject, the cancer being characterized by an elevated amount of mitochondrial DNA (mtDNA) having a deletion of 3926 base pairs within a region of mtDNA between nucleotides 12317 and 16254, with respect to SEQ ID NO. 3, of the human mtDNA genome, the kit comprising:

material for collecting or containing one or more biological samples; and,

at least one pair of PCR primers for detecting the deletion, wherein:
(i) one primer of the at least one pair of primers has a nucleic acid sequence that binds to a splice region of the mtDNA after removal of the deletion; and/or,
(ii) one primer of the at least one pair of primers has a nucleic acid sequence that binds to a region of the deletion comprising a rejoining site after the deletion has recircularized.

In a further aspect, there is provided a kit for quantifying, in a biological sample from a human subject, (a) the amount of mitochondrial DNA (mtDNA) having a deletion of 3926 base pairs within a region of mtDNA between nucleotides 12317 and 16254 of SEQ ID NO. 3 corresponding to the human mtDNA genome, or (b) the amount of a mtDNA deletion comprising 3926 base pairs from a region of mtDNA between nucleotides 12317 and 16254 of SEQ ID NO. 3 corresponding to the human mtDNA genome, the kit comprising:

material for collecting or containing one or more biological samples; and,

at least one pair of PCR primers, wherein:
(i) one primer of the at least one pair of primers has a nucleic acid sequence that binds to a splice region of the mtDNA after removal of the deletion; and/or,
(ii) one primer of the at least one pair of primers has a nucleic acid sequence that binds to a region of the deletion comprising a rejoining site after the deletion has recircularized.

### BRIEF DESCRIPTION OF THE FIGURES

These and other features of the invention will become more apparent in the following detailed description in which reference is made to the appended drawings.

FIG. 1 is a graph showing cycle threshold as related to Example 1.

FIG. 2 shows a ROC curve illustrating the specificity and sensitivity of one embodiment of the present invention.

FIG. 3 is a graph showing cycle threshold as related to Example 2.

FIG. 4 shows a ROC curve illustrating the specificity and sensitivity of another embodiment of the present invention.

FIG. 5 is a schematic diagram showing the design and sequence (SEQ ID NO: 4) of a primer useful for the detection of the 4 kb deletion.

FIG. 6 shows a ROC curve illustrating the specificity and sensitivity of another embodiment of the present invention.

FIG. 7 illustrates normalized qPCR data showing the difference in the amount of the 4 kb deletion in the blood plasma of men with and without prostate cancer (“PCa”).

FIG. 8 shows a ROC curve for Example 4 illustrating sensitivity for prostate cancer detection.

FIG. 9 illustrates normalized qPCR data showing the difference in the amount of the 4 kb deletion in the blood plasma of women with and without breast cancer (“BCa”).

FIG. 10 shows a ROC curve for Example 4 illustrating sensitivity for breast cancer detection.

### DETAILED DESCRIPTION OF THE INVENTION

The present invention provides methods of predicting, diagnosing and monitoring cancer. The methods comprise obtaining one or more biological samples, extracting mitochondrial DNA (mtDNA) from the samples, quantifying the amount of a mitochondrial mutation in the samples and comparing the quantity of the mutation in a sample with a reference value. In this regard, the methods provide a comprehensive tool for determining disease onset and for assessing the predisposition of an individual to cancer. The methods also allow for the monitoring of an individual's risk factors over time and/or for monitoring a patient's response to therapeutic agents and treatment regimes.

### Definitions

Unless defined otherwise, all technical and scientific terms used herein have the same meaning as commonly understood by one of ordinary skill in the art to which this invention belongs.

As used herein, the term “about” refers to an understood variation from the stated value. It is to be understood that such a variation is always included in any given value provided herein, whether or not it is specifically referred to.

As defined herein, “biological sample” refers to a tissue or bodily fluid containing cells from which mtDNA can be obtained. For example, the biological sample can be derived from tissue such as breast or prostate tissue, or from blood, saliva, cerebral spinal fluid, sputa, urine, mucous, synovial fluid, peritoneal fluid, amniotic fluid and the like. The biological sample may be a surgical specimen or a biopsy specimen. The biological sample can be used either directly as obtained from the source or following a pre-treatment to modify the character of the sample. Thus, the biological sample can be pre-treated prior to use by, for example, preparing plasma or serum from blood, disrupting cells, preparing liquids from solid materials, diluting viscous fluids, filtering liquids, distilling liquids, concentrating liquids, inactivating interfering components, adding reagents, and the like.

As used herein, “cycle threshold” (C_{T}) is the point at which target amplification using real-time PCR rises above background, as indicated by a signal such as a fluorescence signal. The C_{T} is inversely related to the quantity of the sequence being investigated.

As used herein, “diagnostic” or “diagnosing” means using the presence or absence of a mutation or combination of mutations as a factor in disease diagnosis or management. The detection of the mutation(s) can be a step in the diagnosis of a disease.

As used herein, “deletion” means removal of a region of mtDNA from a contiguous sequence of mtDNA. Deletions can range in size from one base to thousands of bases or larger.

As used herein, “mitochondrial DNA” or “mtDNA” is DNA present in mitochondria.

As used herein, “mutation” encompasses any modification or change in mitochondrial DNA from the wild type sequence, including without limitation point mutations, transitions, insertions, transversions, translocations, deletions, inversions, duplications, recombinations or combinations thereof. The modification or change of the sequence can extend from a single base change to the addition or elimination of an entire DNA fragment.

As defined herein, “sensitivity” refers to the fraction of true positives (true positive rate) results obtained using the method of the present invention.

As defined herein, “specificity” refers to the fraction of false positives (false positive rate) results obtained using the method of the present invention.

The terms “therapy” and “treatment,” as used interchangeably herein, refer to an intervention performed with the intention of improving a subject's status. The improvement can be subjective or objective and is related to ameliorating the symptoms associated with, preventing the development of, or altering the pathology of a disease. Thus, the terms therapy and treatment are used in the broadest sense, and include the prevention (prophylaxis), moderation, reduction, and curing of a disease, at various stages. Preventing deterioration of a subject's status is also encompassed by the term. Subjects in need of therapy/treatment thus include those already having the disease, as well as those prone to, or at risk of developing, the disease, and those in whom the disease is to be prevented.

### Assays for Predicting, Diagnosing and Monitoring Cancer

### Assay for Detection of Mitochondrial Mutation

Mitochondrial DNA (mtDNA) dynamics are an important diagnostic tool. Mutations in mtDNA are often preliminary indicators of developing disease and may act as biomarkers indicative of risk factors associated with disease onset. As discussed herein, measuring the level of mitochondrial DNA aberration in a biological sample can determine the presence of one or more cancers and identify the potential risk or predisposition of a patient to one or more cancers. Furthermore, measurement of mtDNA at regular intervals can provide health care professionals with a real-time, quantitative monitoring tool for measuring the progression of a patient over time and/or as an assessment for treatment recommendations in order to determine their effectiveness in preventing or treating cancer.

The present invention, therefore, provides methods for predicting, diagnosing or monitoring cancer, comprising obtaining one or more biological samples, extracting mitochondrial DNA (mtDNA) from the samples, and assaying the samples for mitochondrial mutation by: quantifying the amount of an mtDNA aberration in the sample and comparing the level of the aberration with a reference value. As would be understood by those of skill in the art, the reference value is based on whether the method seeks to predict, diagnose or monitor cancer. Accordingly, the reference value may relate to mtDNA data collected from one or more known non-cancerous biological samples, from one or more known cancerous biological samples, and/or from one or more biological samples taken over time. These reference values are used for comparison with the mtDNA data collected from the one or more biological samples wherein, for example, a similar or elevated amount of deletion in the biological sample compared to the reference sample is indicative of a predisposition to or the onset of cancer, or wherein an increasing level of the deletion over time is indicative of cancer onset.

In accordance with an aspect of the invention, the methods for predicting, monitoring and diagnosing cancer comprise an assay for detecting and quantifying one or more mitochondrial mutations. In accordance with one embodiment of the invention, the mutation is an mtDNA deletion. In accordance with another embodiment, the mutation is an mtDNA deletion of 3926 bp of mtDNA (referred to herein as “the 4 kb deletion” or “4 kb sequence”). In accordance with yet another embodiment, the mutation is an mtDNA deletion having the sequence as set forth in SEQ ID NO:1 or SEQ ID NO:2, there being no difference between SEQ ID NO: 1 and SEQ ID NO: 2 when in circular form.

The 4 kb deletion spans approximately nucleotides 12317 and 16254 of the human mtDNA genome. The human mtDNA genome is listed herein as SEQ ID NO:3 (Genbank accession no. AC_000021). The 4 kb deletion is characterized by direct flanking repeats 12 bp in size, with the repeats located at positions 12317-12328 and 16243 to 16254. The repeat sequence is 5′-TGCAACTCCAAA-3′ (SEQ ID NO: 7). Thus, in accordance with one embodiment of the invention, the mutation is an mtDNA deletion of between about residue 12317 and about residue 16254 of the human mtDNA genome.

The inventors have determined, as provided by way of example below, that this deletion is associated with cancer and in particular prostate and breast cancer. Therefore, such deletion provides an accurate biomarker and, therefore, a valuable tool for the detection, diagnosis, or monitoring of cancer in at least these tissues.

The deletion results in the creation of two deletion monomers, one of 4 kb in size (small sublimon) and one of approximately 12.5 kb in size (large sublimon). The occurrence of the deletion may be detected by either identifying the presence of the small sublimon or the large sublimon, the 4 kb or 12.5 kb sequence respectively.

Exemplary methods for assaying the mitochondrial mutation are provided in the Example section. Extraction of mtDNA from a sample may be undertaken using any suitable known method. MtDNA extraction is followed by amplification of all or a region of the mitochondrial genome, and may include sequencing of the mitochondrial genome, as is known in the art and described, for example, in Current Protocols in Molecular Biology (Ausubel et al., John Wiley & Sons, New York, 2007). Likewise, methods for detecting the presence of mutations in the mtDNA can be selected from suitable techniques known to those skilled in the art. For example, analyzing mtDNA can comprise sequencing the mtDNA, amplifying mtDNA by PCR, Southern, Northern, Western South-Western blot hybridizations, denaturing HPLC, hybridization to microarrays, biochips or gene chips, molecular marker analysis, biosensors, melting temperature profiling or a combination of any of the above.

Any suitable means to sequence mitochondrial DNA may be used. Preferably, mtDNA is amplified by PCR prior to sequencing. The method of PCR is well known in the art and may be performed as described in Mullis and Faloona, 1987, Methods Enzymol., 155: 335. PCR products can be sequenced directly or cloned into a vector which is then placed into a bacterial host. Examples of DNA sequencing methods are found in Brumley, R. L. Jr. and Smith, L. M., 1991, Rapid DNA sequencing by horizontal ultrathin gel electrophoresis, Nucleic Acids Res. 19:4121-4126 and Luckey, J. A., et al, 1993, High speed DNA sequencing by capillary gel electrophoresis, Methods Enzymol. 218: 154-172. The combined use of PCR and sequencing of mtDNA is described in Hopgood, R., et al, 1992, Strategies for automated sequencing of human mtDNA directly from PCR products, Biotechniques 13:82-92 and Tanaka, M. et al, 1996, Automated sequencing of mtDNA, Methods Enzymol. 264: 407-421.

Although real-time quantitative PCR methods, as described in the examples below, represent the preferred means for detecting and quantifying the presence or absence of the 4 kb deletion, other methods would be well known to an individual of skill in the art and could be utilized as indicated above. In addition, quantification of the deletion could be made using Bio-Rad's Bioplex™ System and Suspension Array technology. Generally, the method requires amplification and quantification of sequences using any known methods.

The following primer sequences are examples of primers that may be used for the detection of the 4 kb deletion:

“4 forward” (binds to bases 12313-12328
(TTGGTGCAACTCCAAA; SEQ ID NO: 8)/16255-16267
(GCCACCCCTCACC; SEQ ID NO: 9) of the human mtDNA
genome)
(SEQ ID NO: 4)
5′-TTGGTGCAACTCCAAAGCCACCCCTCACC-3′;
“4 reverse” (binds to bases 16391-16409
of the human mtDNA genome)
(SEQ ID NO: 5)
5′-AGGATGGTGGTCAAGGGAC-3′.

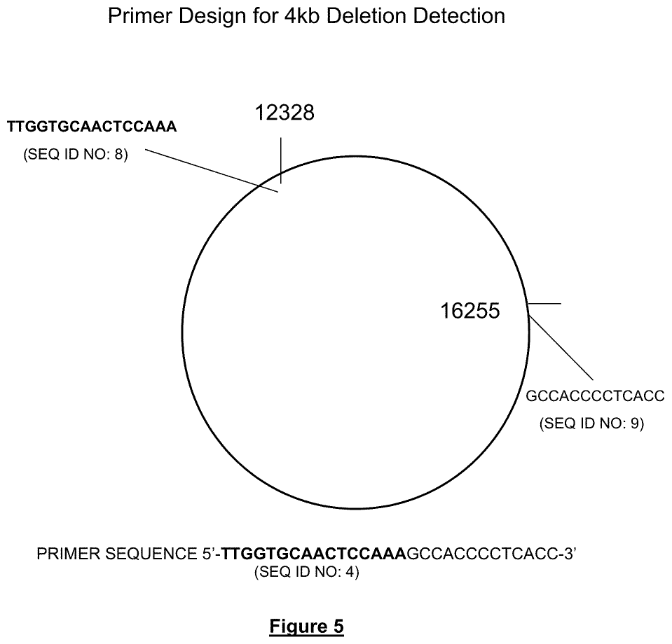

In one embodiment of the present invention, a pair of amplification primers are used to amplify a target region indicative of the presence of the 4 kb deletion. In this embodiment, one of the pair of amplification primers overlaps a spliced region of mtDNA after deletion of the 4 kb sequence has occurred and the mtDNA has reformed as a circular mtDNA molecule (eg. a splice at a position between 12328 and 16255 of the mtDNA genome). Therefore, extension of the overlapping primer can only occur if the 4 kb section is deleted. FIG. 5 is a schematic diagram showing the design and sequence of the primer (ie. SEQ ID NO: 4).

In another embodiment of the present invention, a pair of amplification primers are used to amplify a target region associated with the deleted 4 kb sequence. The deleted 4 kb sequence, upon deletion, may reform as a circular mtDNA molecule. In this embodiment, one of the pair of amplification primers overlaps the rejoining site of the ends of the 4 kb sequence. Thus, an increase in the amount of the 4 kb molecule detected in a sample is indicative of cancer.

In still another embodiment of the present invention, the breakpoint of the deletion is unknown thereby resulting in two possibilities for primer location. In this embodiment, two separate forward primers may be designed to amplify the target region associated with the deleted 4 kb sequence. The following primer sequences are examples of those that may be used for the detection of the 4 kb deletion in this scenario:

### Forward Primers:

Primer A (binds to bases 12313-12328/16255-16267
of the human mtDNA genome)
(SEQ ID NO: 4)
5′-TTGGTGCAACTCCAAAGCCACCCCTCACC-3′;
Primer B (binds to bases 12302-12316 of the
human mtDNA genome)
(SEQ ID NO: 6)
5′-CCCAAAAATTTTGGTGCAACTCCAAAGCCAC-3′

### Reverse Primer:

Primer C (binds to bases 16391-16409 of the human
mtDNA genome)
(SEQ ID NO: 5)
5′-AGGATGGTGGTCAAGGGAC-3′.

As would be understood by a person of skill in the art, the forward primers A or B can be used with reverse primer C to create PCR products that are useful in qPCR assays.

In another aspect, the primers used to detect and amplify the target deletion, in particular the rejoined mtDNA molecule that results after the deletion sequence is removed, may comprise a reverse primer that overlaps the aforementioned splice region, or junction, of the mtDNA molecule. In other words, the splice region includes the junction that results after the 4 kb deletion sequence is removed and the remaining ends of the mtDNA molecule are rejoined. In such case, it will be understood that the forward primer can be of any size, depending on the desired size of the amplicon. In any event, it will be understood that by using a reverse primer that binds to the junction, the resulting amplicon would be indicative of the subject mtDNA deletion regardless of the forward primer that forms the primer pair.

One example of a reverse primer that binds to the mtDNA splice region is:

(SEQ ID NO: 10)
GAG GGG TGG CTT TGG AGT TGC

One example of a forward primer that can be used with the above-noted reverse primer is:

(SEQ ID NO: 11)
AAC AGA GGC TTA CGA CCC CTT

### Biological Sample

The present invention provides for diagnostic tests which involve obtaining or collecting one or more biological samples. In the context of the present invention, “biological sample” refers to a tissue or bodily fluid containing cells from which mtDNA can be obtained. For example, the biological sample can be derived from tissue including, but not limited to, breast, prostate, nervous, muscle, heart, stomach, colon tissue and the like; or from blood, saliva, cerebral spinal fluid, sputa, urine, mucous, synovial fluid, peritoneal fluid, amniotic fluid and the like. The biological sample may be obtained from a cancerous or non-cancerous tissue and may be a surgical specimen or a biopsy specimen.

The biological sample can be used either directly as obtained from the source or following a pre-treatment to modify the character of the sample. Thus, the biological sample can be pre-treated prior to use by, for example, preparing plasma or serum from blood, disrupting cells, preparing liquids from solid materials, diluting viscous fluids, filtering liquids, distilling liquids, concentrating liquids, inactivating interfering components, adding reagents, and the like.

One skilled in the art will understand that more than one sample type may be assayed at a single time (i.e. for the detection of more than one cancer). Furthermore, where a course of collections are required, for example, for the monitoring of risk factors or cancer over time, a given sample may be diagnosed alone or together with other sample taken throughout the test period. In this regard, biological samples may be taken once only, or at regular intervals such as biweekly, monthly, semi-annually or annually.

One of skill will also appreciate that mitochondrial DNA targets are in much greater abundance (approximately 1000 fold greater) than nucleic acid targets and as such sample sizes comprising extremely low yields of nucleic acids would be suitable for use with the present invention.

### Applications for Predicating, Diagnosing and Monitoring Cancer

### Diagnosing and Monitoring Cancer

The prevalence of cancer in most tissue types and age groups necessitates the availability of a tool to not only detect the presence of cancer, but also to monitor the success and appropriateness of preventative measures and therapies being advised to prevent onset, progression and spread of the disease. Measuring the level of mitochondrial DNA deletions in one or more biological samples of an individual can provide initial diagnosis of risk factors, cancer and/or stages of the disease.

The system and method of the present invention, for example, may be used to detect cancer at an early stage, and before any histological abnormalities. Furthermore, sample testing at regular intervals such as biweekly, monthly, semi-annually or annually (or any other suitable interval) can provide health care professionals with a real-time, quantitative monitoring tool to compare against treatment recommendations to determine their effectiveness in preventing or treating the disease.

Turning now to the examples, in one embodiment the present invention may be used for detecting the presence of pre-neoplasia, neoplasia and progression towards potential malignancy of prostate cancer and breast cancer. In one aspect, the present invention involves the detection and quantification of the 4 kb mtDNA deletion for the detection, diagnosis, and/or monitoring of cancer. In this method, mtDNA is extracted from a biological sample (for example body tissue, or body fluids such as urine, prostate massage fluid). The extracted mtDNA is then tested in order to determine the levels (ie. quantity) of the 4 kb deletion in the sample. In tests conducted by the present inventors, the levels of the deletion were found to be elevated in samples obtained from subjects with cancer when compared to samples obtained from subjects without cancer. Based on the information and data supplied below, the inventors have concluded that elevated levels of the 4 kb deletion in human mtDNA is indicative of cancer.

In another embodiment, samples of, for instance prostate tissue, prostate massage fluid, urine or breast tissue, are obtained from an individual and tested over a period of time (eg. years) in order to monitor the genesis or progression of cancer. Increasing levels of the 4 kb deletion over time could be indicative of the beginning or progression of cancer.

One of ordinary skill in the art will appreciate that analysing one or more biological samples from an individual for quantification of a mitochondrial DNA target provides a means for a health care worker to monitor the effectiveness of treatment regimes. One of ordinary skill will also appreciate the utility of mtDNA analysis for use by health care providers in identifying (and providing recommendations for) lifestyle habits, such as poor diet and exercise, or activities that cause over exposure of an individual to known carcinogens (eg. tobacco, pollutants).

Another aspect of the invention provides methods for confirming or refuting the results of a cancer biopsy test from a biopsy sample (eg. prostate or breast cancer), comprising: obtaining non-cancerous tissue from a biopsy sample; and detecting and quantifying the amount of the 4 kb mtDNA deletion in the non-diseased tissue.

### Determining Genetic Predisposition to Cancer

In order to fully evaluate an individual's risk of one or more cancers it is imperative that health care providers are provided with as much information as possible to understand and communicate their patient's risk factors. The utilization of the present invention to determine the level of mtDNA aberration will not only prove helpful in assessing an individual's susceptibility to one or more cancers, it provides a valuable tool to identify patients with greater risk who are potentially in need of more aggressive monitoring and treatment measures.

In this regard, the various examples provided below illustrate a difference in the amount of mtDNA having the 4 kb deletion between samples obtained from subjects having cancer, and subjects without cancer. The amount of the 4 kb deletion was found to be higher in the samples obtained from subjects having cancer. This determination was made by comparing the amount of the 4 kb deletion in the samples from known cancer cells and/or known non-cancer cells.

As such, the inventors determined that screening of biological samples would prove useful in identifying an individual's predisposition to one or more cancers. Thus, in accordance with one embodiment of the present invention there is provided a method for screening individuals for cancer from one or more biological samples comprising: obtaining the one or more samples, and detecting and quantifying the level of the 4 kb mtDNA deletion in the samples. In a specific embodiment of the invention, there is provided a method for screening individuals for prostate or breast cancer from a body fluid or tissue sample comprising; obtaining the body fluid or tissue sample, and detecting and quantifying the level of the 4 kb mtDNA deletion in the body fluid or tissue sample.

Age related accumulation of the 4 kb mtDNA deletion may also predispose an individual to, for example, prostate cancer or breast cancer, which is prevalent in middle aged and older men, and middle aged and older women, respectively. Similarly, an accumulation of the 4 kb mtDNA deletion may be associated with a particular lifestyle based on an individual's diet, exercise habits, and exposure to known carcinogens. Thus, in accordance with one aspect of the invention, a method is provided wherein regular cancer screening may take place by monitoring over time the amount of the 4 kb deletion in one or more biological samples, non-limiting examples of which include breast and prostate tissues or body fluids such as prostate massage fluid, or urine.

### Evaluation of Therapeutic Agents

The method of the present invention may also be used for screening potential therapeutic agents for use in cancer treatment or for monitoring the therapeutic effect of such agents. The method of the present invention may be used to measure various biomarkers associated with the cancers identified herein. The ability to assess the level of DNA damage in any biological sample at any time point provides the foundation for a unique and informative screening test for an individual's health and to assess the safety and efficacy of existing and new therapeutic agents and treatment regimes. Furthermore, by identifying the specific genetic changes underlying a subject's state of health, it may be readily determined whether and to what extent a patient will respond to a particular therapeutic agent or regime.

### Kits

The present invention provides diagnostic/screening kits for use in a clinical environment. Such kits could not only include one or more sampling means, but other materials necessary for the identification of mtDNA mutations.

The kits can optionally include reagents required to conduct a diagnostic assay, such as buffers, salts, detection reagents, and the like. Other components, such as buffers and solutions for the isolation and/or treatment of a biological sample, may also be included in the kit. One or more of the components of the kit may be lyophilised and the kit may further comprise reagents suitable for the reconstitution of the lyophilised components.

Where appropriate, the kit may also contain reaction vessels, mixing vessels and other components that facilitate the preparation of the test sample. The kit may also optionally include instructions for use, which may be provided in paper form or in computer-readable form, such as a disc, CD, DVD or the like.

In one aspect of the invention there is provided a kit for diagnosing cancer comprising means for extraction of mtDNA, primers, reagents and instructions.

In another aspect of the invention there is provided a kit for diagnosing cancer, for example prostate or breast cancer, comprising means for extraction of mtDNA, primers having the nucleic acid sequences recited in SEQ ID NOs: 4 and 5, reagents and instructions.

In another aspect of the invention there is provided a kit for diagnosing cancer, for example prostate or breast cancer, comprising means for extraction of mtDNA, primers having the nucleic acid sequences recited in SEQ ID NOs: 6 and 5, reagents and instructions.

To gain a better understanding of the invention described herein, the following examples are set forth. It will be understood that these examples are intended to describe illustrative embodiments of the invention and are not intended to limit the scope of the invention in any way.

### EXAMPLES

### Example 1: Association of Prostate Cancer with 4 kb Deletion in Human mtDNA

Urine samples were collected from five patients who had been diagnosed with prostate cancer and five who had a needle biopsy procedure which was unable to detect prostate malignancy. These samples were collected following a digital rectal exam (DRE) to facilitate the collection of prostate cells.

Upon receipt of the samples a 5 ml aliquot was removed and then 2 mls were centrifuged at 14,000×g to form a pellet. The supernatant was removed and discarded.

Pellets were resuspended in 200 ul phosphate buffered saline solution. Both the resuspended pellet and the whole urine sample were subjected to a DNA extraction procedure using the QiaAMP DNA Mini Kit (Qiagen P/N 51304) according to the manufacturer's directions.

The resulting DNA extracts were then quantified using a NanoDrop ND-1000 Spectrophotometer and normalized to a concentration of 0.1 ng/ul.

Samples were analyzed by quantitative real-time PCR with the 4 kb deletion specific primers according to the following:

1X iQ SYBR Green Supermix (Bio-Rad product no.
170-8880)
100 nmol forward primer
(SEQ ID NO: 4)
(5′-TTGGTGCAACTCCAAAGCCACCCCTCACC-3′)
100 nmol reverse primer
(SEQ ID NO: 5)
(5′-AGGATGGTGGTCAAGGGAC-3′)
1 ng template DNA in a 25 ul reaction

Reactions were cycled on an Opticon 2 DNA Engine (Bio-Rad Canada) according to the following protocol:
1. 95° C. for 3 minutes
2. 95° C. for 30 seconds
3. 69° C. for 30 seconds
4. 72° C. for 30 seconds
5. Plate Read
6. Repeat steps 2-5 44 times
7. 72° C. for 10 minutes
8. Melting Curve from 50° C. to 105° C., read every 1° C., hold for 3 seconds
9. 10° C. Hold

### Results

Results from the urine pellet did not yield significant differences in the mean cycle threshold observed or a useful cutoff point. However, the results from the whole urine sample did yield significant differences as provided below.

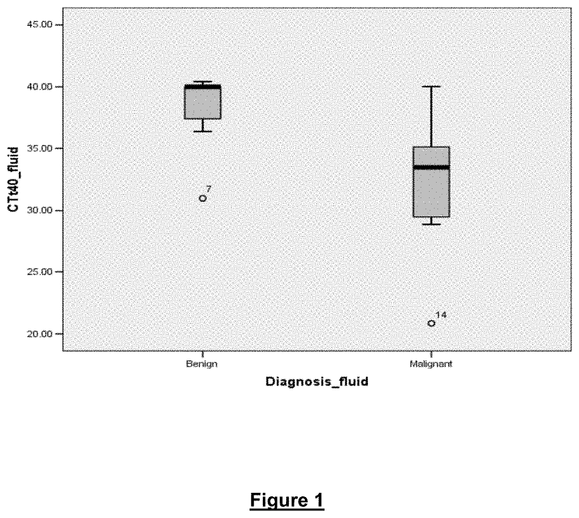

Tables 1 and 2, and FIG. 1 show the difference in the mean C_{T} scores for urine samples from subjects having prostate malignant tissue and benign tissue at the 0.04 significance level.

TABLE 1
Mean Values for C_{T} scores: Urine Samples

| | | | Std. | Std. Error |
|---|---|---|---|---|
| | N | Mean | Deviation | Mean |
| Benign | 7 | 38.0357 | 3.40974 | 1.288876 |
| Malignant | 7 | 31.9300 | 6.12583 | 2.31534 |

TABLE 2
Significance Test for Mean C_{T} scores
Independent Samples Test

| | | | Test for Equality Means | | | | | | |
|---|---|---|---|---|---|---|---|---|---|
| | Levene's Test for | | | | | | | 95% Confidence Interval | |
| | Equality of Variances | | | | Sig. | Mean | Std. Error | of the Difference | |
| CTt40 fluid | F | Sig. | t | df | (2-tailed) | Diff. | Diff. | Lower | Upper |
| Equal variances | 1.707 | .216 | 2304 | 12 | .040 | 610571 | 264985 | .33218 | 11.87925 |
| assumed | | | | | | | | | |
| Equal variances | | | 2304 | 9392 | .046 | 610571 | 264985 | .14927 | 12.06215 |
| not assumed | | | | | | | | | |

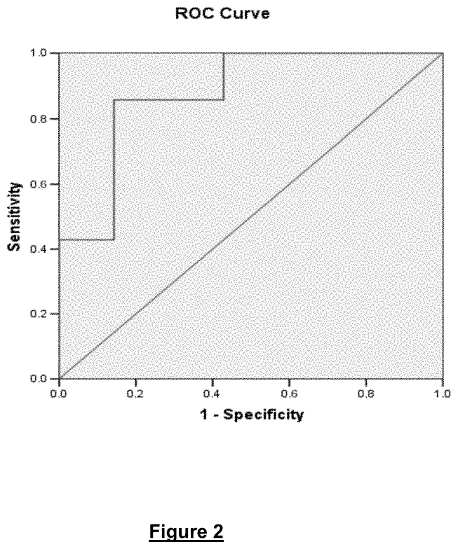

Tables 3 and 4, and FIG. 2 illustrate that when using a cut-off cycle threshold of 36.255 the sensitivity of the assay for prostate cancer is 86% and the specificity is 86%.

FIG. 2 is a Receiver Operating Characteristic (ROC) curve illustrating the specificity and sensitivity of the 4 kb mtDNA deletion as a marker for prostate cancer when testing urine. These results were obtained using a cutoff C_{T} of 36.255. The sensitivity of the marker at this C_{T} is 86%, while the specificity is 86%.

The determination of the cutoff C_{T} of 36.255 is shown in Table 3. The results listed in Table 3 show that a cutoff C_{T} of 36.255 provided the highest sensitivity and specificity.

The accuracy of the test depends on how well the test separates the group being tested into those with and without the prostate cancer. Accuracy is measured by the area under the ROC curve. Table 4 shows the calculation of the area under the curve for the present example.

TABLE 3
Determination of Specificity and
Sensitivity

| Positive if ≤ ^{a} | Sensitivity | 1-specificity |
|---|---|---|
| 19.86 | .000 | .000 |
| 24.87 | .143 | .000 |
| 29.48 | .286 | .000 |
| 30.54 | .429 | .000 |
| 32.235 | .429 | .143 |
| 33.77 | .571 | .143 |
| 35.11 | .714 | .143 |
| 36.255 | .857 | .143 |
| 37.415 | .857 | .286 |
| 39.23 | .857 | .429 |
| 39.995 | 1.000 | .429 |
| 40.21 | 1.000 | .857 |
| 41.42 | 1.000 | 1.000 |

^{a}-the smallest cutoff value is the minimum observed test value minus 1 and the largest cutoff value is the maximum observed test value plus 1. All the other cutoff values are the averages of two consecutive ordered observed test values.

TABLE 4
Results Showing Area Under the ROC Curve

| | | | Asymptotic 95% | |
|---|---|---|---|---|
| | Std. | Asymptotic | Confidence Interval | |
| Area | Error ^{a} | Sig. ^{b} | Lower bound | Upper bound |
| .878 | .096 | .018 | .689 | 1.066 |

Notes:
^{a}-under the non-parametric assumption
^{b}-null hypothesis: true area = 0.5

### Example 2: Association of Breast Cancer with 4 kb Deletion in Human mtDNA

Twenty breast tissue samples were collected, ten of which were malignant and ten of which had benign breast disease or no abnormalities. These samples were formalin-fixed paraffin embedded and 20 micron sections of each were cut into individual sample tubes for extraction according to the manufacturer's protocol for the QiaAMP DNA Mini Kit (Qiagen P/N 51304). DNA was then quantified using a Nanodrop ND-1000 and normalized to a concentration of 2 ng/ul.

Samples were then assayed for the levels of the 4 kb deletion by quantitative real-time PCR using the following protocol:

X iQ SYBR Green Supermix (Bio-Rad product no. 170-8880)

175 nmol forward primer (5′-TTGGTGCAACTCCAAAGCCACCCCTCACC-3′) (SEQ ID NO: 4)

175 nmol reverse primer (5′-AGGATGGTGGTCAAGGGAC-3′) (SEQ ID NO: 5)

20 ng template DNA in a 25 ul reaction

Reactions were cycled on an Opticon 2 DNA Engine (Bio-Rad Canada) according to the following protocol:
1. 95° C. for 3 minutes
2. 95° C. for 30 seconds
3. 70° C. for 30 seconds
4. 72° C. for 30 seconds
5. Plate Read
6. Repeat steps 2-5 44 times
7. 72° C. for 10 minutes
8. Melting Curve from 50° C. to 105° C., read every 1° C., hold for 3 seconds
9. 10° C. Hold

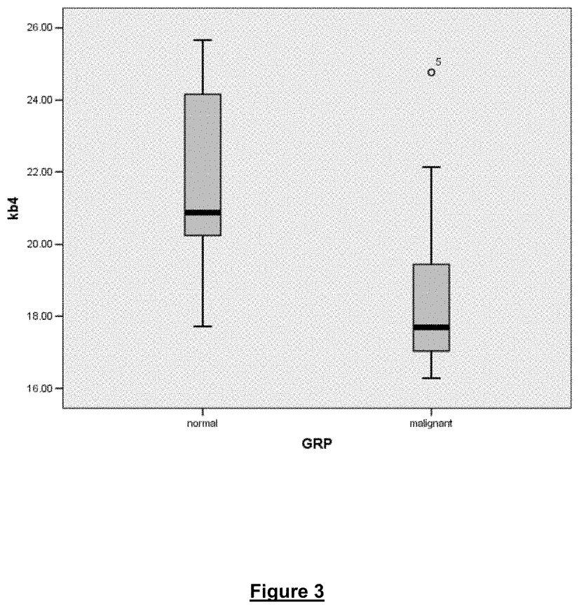

Tables 5 and 6, and FIG. 3 show the difference in the mean C_{T} scores for breast tissue samples from subjects having malignant breast tissue and benign breast tissue at the 0.065 level.

TABLE 5
Mean Values for CT scores: Breast Tissue Samples

| Group | N | Mean | Std. Dev. | Std. Error Mean |
|---|---|---|---|---|
| Normal | 9 | 21.5278 | 2.71939 | .90646 |
| Malignant | 9 | 18.9089 | 2.89126 | .96375 |

TABLE 6
Significance Test for Mean C_{T} scores

| | | | Test for Equality Means | | | | | | |
|---|---|---|---|---|---|---|---|---|---|
| | Levene's Test for | | | | | | | 95% Confidence Interval | |
| | Equality of Variances | | | | Sig. | Mean | Std. Error | of the Difference | |
| CTt40 fluid | F | Sig. | t | df | (2-tailed) | Diff. | Diff. | Lower | Upper |
| Equal variances | .007 | .934 | 1.979 | 16 | .065 | 2.61889 | 1.32306 | -.18588 | 5.42366 |
| assumed | | | | | | | | | |
| Equal variances | | | 1.979 | 15.94 | .065 | 2.61889 | 1.32306 | -.18674 | 5.42452 |
| not assumed | | | | | | | | | |

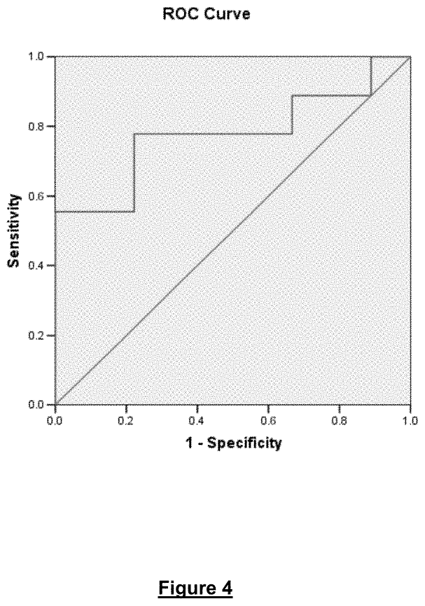

Tables 7 and 8, and FIG. 4 illustrate that when using a cut-off cycle threshold of 19.845 the sensitivity of the assay for breast cancer is 78% and the specificity is 78%.

FIG. 4 is an ROC curve illustrating the specificity and sensitivity of the 4 kb mtDNA deletion as a marker for breast cancer when testing breast tissue. These results were obtained using a cutoff C_{T} of 19.845. The sensitivity of the marker at this C_{T} is 78%, while the specificity is 78%.

The determination of the cutoff C_{T} of 19.845 is shown in Table 7. The results listed in Table 7 show that a cutoff C_{T} of 19.845 provided the highest sensitivity and specificity.

The accuracy of the test depends on how well the test separates the group being tested into those with and without the breast cancer. Accuracy is measured by the area under the ROC curve. Table 8 shows the calculation of the area under the curve for the present example.

TABLE 7
Determination of Specificity and Sensitivity

| Positive if ≤ ^{a} | Sensitivity | 1-specificity |
|---|---|---|
| 15.28 | .000 | .000 |
| 16.305 | .111 | .000 |
| 16.69 | .222 | .000 |
| 17.075 | .333 | .000 |
| 17.4 | .444 | .000 |
| 17.71 | .556 | .000 |
| 18.0 | .556 | .111 |
| 18.835 | .556 | .222 |
| 19.415 | .667 | .222 |
| 19.845 | .778 | .222 |
| 20.475 | .778 | .333 |
| 10.79 | .778 | .444 |
| 21.38 | .778 | .556 |
| 22.005 | .778 | .667 |
| 23.145 | .889 | .667 |
| 24.19 | .889 | .778 |
| 24.49 | .889 | .889 |
| 25.21 | 1.00 | .889 |
| 26.66 | 1.00 | 1.00 |

^{a}-the smallest cutoff value is the minimum observed test value minus 1 and the largest cutoff value is the maximum observed test value plus 1. All the other cutoff values are the averages of two consecutive ordered observed test values.

TABLE 8
Results Showing Area Under the ROC Curve

| | | | Asymptotic 95% | |
|---|---|---|---|---|
| | Std. | Asymptotic | Confidence Interval | |
| Area | Error ^{a} | Sig. ^{b} | Lower bound | Upper bound |
| .778 | .117 | .047 | .548 | 1.008 |

### Example 3: Association of Prostate Cancer with 4 kb Deletion in Human mtDNA Using Needle Biopsy Samples

Prostate needle biopsy specimens were obtained from 19 individuals, 9 without prostate cancer and 10 with prostate cancer. Needle biopsy tissues were formalin-fixed paraffin embedded (FFPE) as is standard in the clinical diagnostic setting. 10 micron sections of each biopsy were deposited directly into centrifuge tubes and the DNA was extracted using the QiaAMP DNA Mini Kit (Qiagen, p/n 51306). DNA extracts were quantified by absorbance at 260 nm using a NanoDrop ND-1000 Spectrophotometer. Yields ranged from 347 ng to 750 ng. These samples were diluted to 2 ng/ul and amplification reactions setup according to Table 9 and the following:

TABLE 9
Reagents and Concentrations for Amplification
Reaction

| | Final |
|---|---|
| | Concen- |
| Reagent | tration |
| iQ SYBR Green Supermix | 1X |
| (Bio-Rad Laboratories, p/n 170-8882) | |
| Forward Primer 12303-12316/16243-16259F | 175 nmol |
| 5′-CCCAAAAATTTTGGTGCAACTCCAAAGCCAC-3′ | |
| (SEQ ID NO: 6) | |
| Reverse Primer 16410R | 175 nmol |
| 5′-AGGATGGTGGTCAAGGGAC-3′ | |
| (SEQ ID NO: 5) | |
| DNA extract | 0.8 ng/ul |

Nuclease-free water was added to a final reaction volume of 25 ul. Amplifications were carried out on a DNA Engine Chromo4 Real Time PCR Instrument (Bio-Rad Laboratories) according the following cycling conditions:
1) 95° C. for 3 minutes
2) Followed by 45 cycles of
3) 95° C. for 30 seconds
4) 69° C. for 30 seconds
5) 72° C. for 30 seconds
6) Plate Read

### Then

7) 72° C. for 10 minutes
8) Melting Curve 50° C.-105° C. reading every 1° C., hold for 3 seconds
9) 4° C. Hold

Results, shown in Table 10, demonstrate that those individuals with prostate cancer have a lower C_{T} value and therefore higher levels of the 4 kb deletion in prostate tissue than do those without prostate cancer. Patients with prostate cancer have an average C_{T} value of 30.7 while the patients without prostate cancer have an average C_{T} value of 36.4. This difference of 5.7 C_{T} corresponds to nearly 100 fold greater 4 kb deletion levels in the group with prostate malignancy than in the group without.

TABLE 10
Patient Diagnosis and Associated C_{T} Score

| Patient Number and Diagnosis | C(t) |
|---|---|
| CUG 1301 Malignant | 25.7 |
| CUG 1268 Malignant | 27.7 |
| CUG RN 345 Normal | 28.3 |
| CUG 1272 Malignant | 28.8 |
| CUG 1375 Malignant | 29.1 |
| CUG 1259 Malignant | 29.1 |
| CUG 1381 Malignant | 30.2 |
| CUG RN 82 Normal | 30.5 |
| CUG 1372 Malignant | 30.9 |
| CUG 1085 C T1 Normal | 31.5 |
| CUG 1317 Malignant | 31.7 |
| CUG 1377 F Normal | 33.6 |
| CUG 1365 B Normal | 34.6 |
| CUG 1370 Malignant | 35.9 |
| CUG RN 405 Normal | 37.5 |
| CUG 1366 Malignant | 37.9 |
| CUG RN 701 Normal | 41.7 |
| CUG RN 420 Normal | 45 |
| CUG RN 373 Normal | 45 |

Tables 11 and 12 show the difference in the mean C_{T} scores for prostate tissue samples from subjects having normal and malignant prostate tissue.

TABLE 11
Mean Values for C_{T} Score: Prostate Needle
Biopsy Tissue

| Group | N | Mean | Std. Dev. | Std. Error Mean |
|---|---|---|---|---|
| Normal | 9 | 36.4111 | 6.25229 | 2.08410 |
| Malignant | 10 | 30.7 | 3.69534 | 1.16857 |

TABLE 12
Significance Test for C_{T} Scores

| | | | Test for Equality Means | | | | | | |
|---|---|---|---|---|---|---|---|---|---|
| | Levene's Test for | | | | | | | 95% Confidence Interval | |
| | Equality of Variances | | | | Sig. | Mean | Std. Error | of the Difference | |
| CTt40 fluid | F | Sig. | t | df | (2-tailed) | Diff. | Diff. | Lower | Upper |
| Equal variances | 4.426 | .051 | 2.455 | 17 | .025 | 5.71111 | 2.32589 | .80391 | 10.61831 |
| assumed | | | | | | | | | |
| Equal variances | | | 2.390 | 12.705 | .033 | 5.71111 | 2.38935 | .53701 | 10.88522 |
| not assumed | | | | | | | | | |

Table 13 and FIG. 6 illustrate that when using a cutoff of C_{T} 32.65 the sensitivity and specificity of correctly diagnosing these patients is 80% and 67% respectively.

TABLE 13
Determination of Specificity and
Sensitivity

| Positive if ≤ ^{a} | Sensitivity | 1-specificity |
|---|---|---|
| 24.7 | .000 | .000 |
| 26.7 | .100 | .000 |
| 28.0 | .200 | .000 |
| 28.55 | .200 | .111 |
| 28.95 | .300 | .111 |
| 29.65 | .500 | .111 |
| 30.35 | .600 | .111 |
| 30.7 | .600 | .222 |
| 31.2 | .700 | .222 |
| 31.6 | .700 | .333 |
| 32.65 | .800 | .333 |
| 34.1 | .800 | .444 |
| 32.25 | .800 | .556 |
| 36.7 | .900 | .556 |
| 37.7 | .900 | .667 |
| 39.8 | 1.000 | .667 |
| 43.35 | 1.000 | .778 |
| 46.0 | 1.000 | 1.000 |

### Example 4: Confirmation of Detection of 4 kb Deletion in Blood Samples

Test were conducted to confirm that the use of blood is viable as a test sample for the methods described herein for detecting the 4 kb mtDNA deletion. The data from these tests is discussed below.

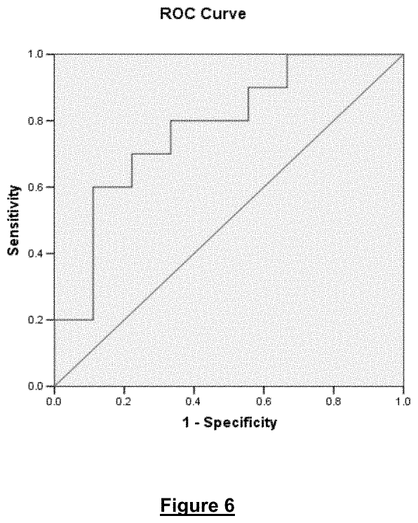

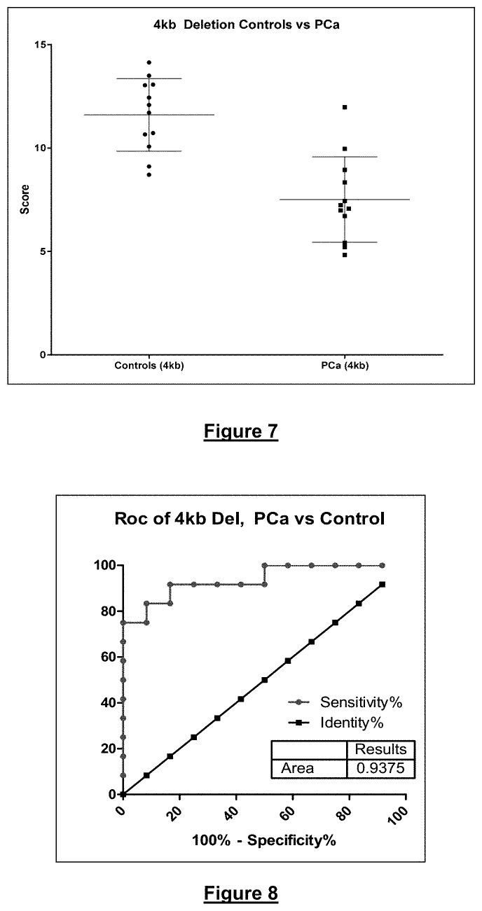

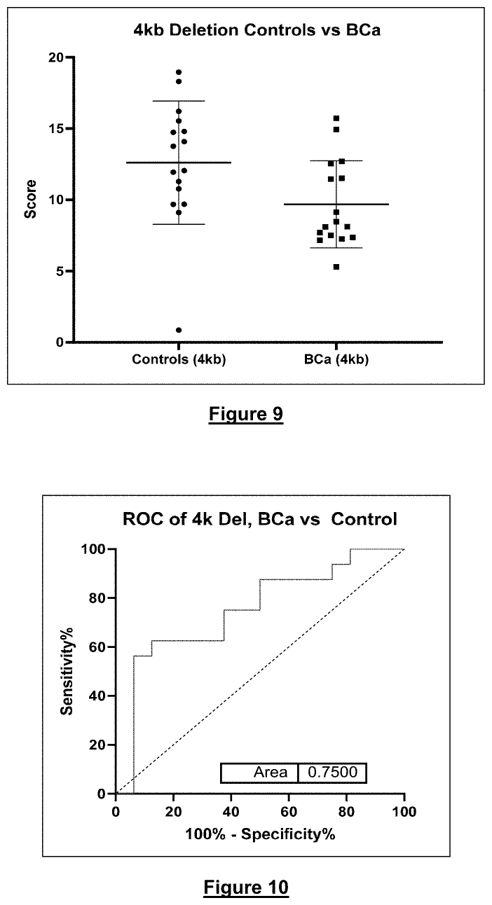

In these tests, blood samples from individuals both with and without breast cancer or prostate cancer were analyzed for the presence of the 4 kb deletion and the results are illustrated in FIGS. 7 to 10. As will be understood, the testing for breast cancer was conducted using samples from female subjects and the testing for prostate cancer was conducted using samples from male subjects. FIGS. 7 and 9 illustrate that the present methods were able to detect breast and prostate cancers using blood samples from the respective subjects. Specifically, FIGS. 7 and 9 illustrate sample scores calculated by taking the difference in qPCR Cq values between the deletion marker and a nuclear control marker (which is not affected by a disease state). Thus, a lower score indicates a greater presence of the deletion in the sample. The receiver operating characteristic curves (ROC curves) are provided in FIGS. 8 and 10, respectively.

Data from this example is provided below in Tables 14 and 15.

TABLE 14
Table showing the performance of the 4 kb deletion biomarker to distinguish
between blood plasma from men with and without prostate cancer at various cut-offs

| Cut-off | Sensitivity % | 95% CI | Specificity % | 95% CI |
|---|---|---|---|---|
| <5.020 | 8.333 | 0.2108% to 38.48% | 100 | 73.54% to 100.0% |
| <5.315 | 16.67 | 2.086% to 48.41% | 100 | 73.54% to 100.0% |
| <6.065 | 25 | 5.486% to 57.19% | 100 | 73.54% to 100.0% |
| <6.850 | 33.33 | 9.925% to 65.11% | 100 | 73.54% to 100.0% |
| <7.030 | 41.67 | 15.17% to 72.33% | 100 | 73.54% to 100.0% |
| <7.155 | 50 | 21.09% to 78.91% | 100 | 73.54% to 100.0% |
| <7.340 | 58.33 | 27.67% to 84.83% | 100 | 73.54% to 100.0% |
| <7.890 | 66.67 | 34.89% to 90.08% | 100 | 73.54% to 100.0% |
| <8.525 | 75 | 42.81% to 94.51% | 100 | 73.54% to 100.0% |
| <8.825 | 75 | 42.81% to 94.51% | 91.67 | 61.52% to 99.79% |
| <9.025 | 83.33 | 51.59% to 97.91% | 91.67 | 61.52% to 99.79% |
| <9.535 | 83.33 | 51.59% to 97.91% | 83.33 | 51.59% to 97.91% |
| <10.02 | 91.67 | 61.52% to 99.79% | 83.33 | 51.59% to 97.91% |
| <10.37 | 91.67 | 61.52% to 99.79% | 75 | 42.81% to 94.51% |
| <10.69 | 91.67 | 61.52% to 99.79% | 66.67 | 34.89% to 90.08% |
| <11.22 | 91.67 | 61.52% to 99.79% | 58.33 | 27.67% to 84.83% |
| <11.84 | 91.67 | 61.52% to 99.79% | 50 | 21.09% to 78.91% |
| <12.03 | 100 | 73.54% to 100.0% | 50 | 21.09% to 78.91% |
| <12.26 | 100 | 73.54% to 100.0% | 41.67 | 15.17% to 72.33% |
| <12.74 | 100 | 73.54% to 100.0% | 33.33 | 9.925% to 65.11% |
| <13.06 | 100 | 73.54% to 100.0% | 25 | 5.486% to 57.19% |
| <13.29 | 100 | 73.54% to 100.0% | 16.67 | 2.086% to 48.41% |
| <13.82 | 100 | 73.54% to 100.0% | 8.333 | 0.2108% to 38.48% |

TABLE 15
Table showing the performance of the 4 kb deletion biomarker to distinguish between
blood plasma from women with and without breast cancer at various cut-offs

| Cut-off | Sensitivity % | 95% CI | Specificity % | 95% CI |
|---|---|---|---|---|
| <3.075 | 0 | 0.000% to 19.36% | 93.75 | 71.67% to 99.68% |
| <6.230 | 6.25 | 0.3206% to 28.33% | 93.75 | 71.67% to 99.68% |
| <7.215 | 12.5 | 2.221% to 36.02% | 93.75 | 71.67% to 99.68% |
| <7.315 | 18.75 | 6.592% to 43.01% | 93.75 | 71.67% to 99.68% |
| <7.440 | 25 | 10.18% to 49.50% | 93.75 | 71.67% to 99.68% |
| <7.605 | 31.25 | 14.16% to 55.60% | 93.75 | 71.67% to 99.68% |
| <7.900 | 37.5 | 18.48% to 61.36% | 93.75 | 71.67% to 99.68% |
| <8.105 | 43.75 | 23.10% to 66.82% | 93.75 | 71.67% to 99.68% |
| <8.285 | 50 | 28.00% to 72.00% | 93.75 | 71.67% to 99.68% |
| <8.785 | 56.25 | 33.18% to 76.90% | 93.75 | 71.67% to 99.68% |
| <9.125 | 56.25 | 33.18% to 76.90% | 87.5 | 63.98% to 97.78% |
| <9.410 | 62.5 | 38.64% to 81.52% | 87.5 | 63.98% to 97.78% |
| <9.685 | 62.5 | 38.64% to 81.52% | 81.25 | 56.99% to 93.41% |
| <10.23 | 62.5 | 38.64% to 81.52% | 75 | 50.50% to 89.82% |
| <11.03 | 62.5 | 38.64% to 81.52% | 68.75 | 44.40% to 85.84% |
| <11.37 | 62.5 | 38.64% to 81.52% | 62.5 | 38.64% to 81.52% |
| <11.49 | 68.75 | 44.40% to 85.84% | 62.5 | 38.64% to 81.52% |
| <11.73 | 75 | 50.50% to 89.82% | 62.5 | 38.64% to 81.52% |
| <12.00 | 75 | 50.50% to 89.82% | 56.25 | 33.18% to 76.90% |
| <12.30 | 75 | 50.50% to 89.82% | 50 | 28.00% to 72.00% |
| <12.62 | 81.25 | 56.99% to 93.41% | 50 | 28.00% to 72.00% |
| <13.23 | 87.5 | 63.98% to 97.78% | 50 | 28.00% to 72.00% |
| <13.92 | 87.5 | 63.98% to 97.78% | 43.75 | 23.10% to 66.82% |
| <14.41 | 87.5 | 63.98% to 97.78% | 37.5 | 18.48% to 61.36% |
| <14.77 | 87.5 | 63.98% to 97.78% | 31.25 | 14.16% to 55.60% |
| <14.87 | 87.5 | 63.98% to 97.78% | 25 | 10.18% to 49.50% |
| <15.24 | 93.75 | 71.67% to 99.68% | 25 | 10.18% to 49.50% |
| <15.63 | 93.75 | 71.67% to 99.68% | 18.75 | 6.592% to 43.01% |
| <15.97 | 100 | 80.64% to 100.0% | 18.75 | 6.592% to 43.01% |
| <17.26 | 100 | 80.64% to 100.0% | 12.5 | 2.221% to 36.02% |
| <18.63 | 100 | 80.64% to 100.0% | 6.25 | 0.3206% to 28.33% |

Although the invention has been described with reference to certain specific embodiments, various modifications thereof will be apparent to those skilled in the art without departing from the spirit and scope of the invention as outlined in the claims appended hereto. All such modifications as would be apparent to one skilled in the art are intended to be included within the scope of the following claims. All documents recited in the present application are incorporated herein by reference.

### REFERENCES

Birch-Machin M A, Online Conference Report (Sunburnt DNA), International Congress of Biochemistry and Molecular Biology, New Scientist, 2000(a).
Birch-Machin M A, Taylor R W, Cochran B, Ackrell B A C, Tumbull D M. Ann Neurol 48: 330-335, 2000(b).
Birch-Machin, M. A. (2000). Mitochondria and skin disease. Clin Exp Dermatol, 25, 141-6.
Brown, M. D., et al., Am J. Humn Genet, 60: 381-387, 1997.
Bogliolo, M, et al., Mutagenesis, 14: 77-82, 1999.
Chinnery P F and Turnbull D M., Lancet 354 (supplement 1): 17-21, 1999.
Huoponen, Kirsi, Leber hereditary optic neuropathy: clinical and molecular genetic findings, Neurogenetics (2001) 3: 119-125.
Hayward S W, Grossfeld G D, Tlsty T D, Cunha G R., Int J Oncol 13:35-47, 1998.
Huang G M, Ng W L, Farkas J, He L, Liang H A, Gordon D, Hood R., Genomics 59(2):178-86, 1999.
Konishi N, Cho M, Yamamoto K, Hiasa Y. Pathol. Int. 47:735-747, 1997.
Landis S H, Murray T, Bolden S, Wngo P A. Cancer J. Clin. 49:8-31.
Lee H C, Lu C Y, Fahn H J, Wei YHu. Federation of European Biochemical Societies, 441:292-296, 1998.
Naviaux, R K., Mitochondrial Disease—Primary Care Physican's Guide. Psy-Ed. Corp D/B/A Exceptional Parents Guide: 3-10, 1997.
Parrella P, Xiao Y, Fliss M, Sanchez-Cespedes M, Mazzarelli P, Rinaldi M, Nicol T, Gabrielson E, Cuomo C, Cohen D, Pandit S, Spencer M, Rabitti C, Fazio V M, Sidransky D: Detection of mitochondrial DNA mutations in primary breast cancer and fine-needle aspirates. Cancer Res 2001, 61:7623-7626.
Polyak Y, et al., Nature Genet. 20 (3):291-293, 1998.
Seidman, M. D. et al., Arch. Otolaryngol Head Neck Surg., 123: 1039-1045, 1997.
Sherrat E J, Thomas A W, Alcolado J C., Clin. Sci. 92:225-235, 1997.
Shoffner J M, Brown M D, Torroni A, Lott M T, Cabell M F, Mirra S S, Beal M F, Yang C, Gearing M, Salvo R, Watts R L, Juncos J L, Hansen L A, Crain B J, Fayad M, Reckford C L, and Wallace D C., Genomics 17: 171-184, 1993.
SpringNet—CE Connection: Screening, Diagnosis: Improving Primary Care Outcomes. Website: http://www.springnet.com/ce/j803a.htm.
Taniike, M. et al., BioChem BioPhys Res Comun, 186: 47-53, 1992.
Valnot, Isabelle, et al., A mitochondrial cytochrome b mutation but no mutations of nuclearly encoded subunits in ubiquinol cytochrome c reductase (complex III) deficiency, Human Genetics (1999) 104: 460-466.
von Wurmb, N, Oehmichen, M, Meissner, C., Mutat Res. 422:247-254, 1998.
Wallace et al., Mitochondiral DNA MUtatio Assoicated with Leber's Hereditary Optic Neuropathy, Science, 1427-1429.
Wei Y H. Proceedings of the Nat. Sci. Council of the Republic of China April 22(2):5567, 1998.
Woodwell D A. National Ambulatory Medical Care Survey: 1997 Summary. Advance data from vital and health statistics; no. 305. Hyattsville, Md.: National Center for Health Statistics. 1999.
Yeh, J. J., et al., Oncogene Journal, 19: 2060-2066, 2000.
Zhang et al., Multiple mitochondiral DNA deletions in an elderly human individual, FEBS Lett, 297, 34-38 1992.
Zhang, C., et al., BioChem. BioPhys. Res. Comun., 195: 1104-1110, 1993.

We claim:

1. A method of detecting breast or prostate cancer, or a genetic predisposition to breast or prostate cancer, in a human subject, the cancer being characterized by an elevated amount of mitochondrial DNA (mtDNA) having a deletion of 3926 base pairs within a region of mtDNA between nucleotides 12317 and 16254, with respect to SEQ ID NO. 3, of the human mtDNA genome, the method comprising:
a) contacting mtDNA extracted from a biological sample from the subject with a pair of PCR primers that specifically bind to a sequence of mtDNA having a spliced region after removal of the deletion, wherein one primer of the pair of primers has the nucleotide sequence as set forth in SEQ ID NO: 10;
b) quantifying the amount of mtDNA having the deletion by quantifying the amount of mtDNA bound to the primers; and,
c) detecting said cancer or said predisposition to cancer where the quantified amount of mtDNA having the deletion is elevated in relation to at least one known reference value.

2. The method of claim 1, wherein the other primer of the pair of primers has the nucleotide sequences as set forth in SEQ ID NO: 11.

3. The method of claim 1, wherein the pair of primers are amplification primers.

4. The method of claim 3, wherein the step (b) comprises amplifying the region of mtDNA bound to the primers.

5. The method of claim 4, wherein step (b) is conducted using real-time PCR.

6. The method of claim 1, wherein the deletion has the nucleotide sequence as set forth in SEQ ID NO: 1 or SEQ ID NO: 2.

7. The method of claim 1, wherein the at least one known reference value is the amount of mtDNA having the deletion in a reference sample of mtDNA from known non-cancerous tissue or body fluid.

8. The method of claim 7, wherein the biological sample is a tissue or bodily fluid containing cellular material from breast or prostate tissue.

9. A method of quantifying, in a biological sample from a human subject, the amount of mitochondrial DNA (mtDNA) having a deletion of 3926 base pairs within a region of mtDNA between nucleotides 12317 and 16254 of SEQ ID NO. 3 corresponding to the human mtDNA genome, the method comprising:
a) contacting the biological sample with a pair of PCR primers, wherein one primer of the pair of primers specifically binds to a region of the mtDNA having a spliced region after removal of the deletion; and,
b) amplifying and quantifying the amount of mtDNA having the deletion using real-time PCR.

10. The method of claim 9, wherein the one primer has the nucleotide sequence as set forth in SEQ ID NO: 10.

11. The method of claim 10, wherein the other primer of the pair of primers has the nucleotide sequence as set forth in SEQ ID NO: 11.

12. The method of claim 9, wherein the deletion has the nucleotide sequence as set forth in SEQ ID NO: 1 or SEQ ID NO: 2.

13. The method of claim 9, wherein the biological sample is a tissue or bodily fluid containing cellular material from breast or prostate tissue.

14. A method of quantifying, in a biological sample from a human subject, the amount of a mitochondrial DNA (mtDNA) deletion, wherein the deletion comprises 3926 base pairs from a region of mtDNA between nucleotides 12317 and 16254 of SEQ ID NO. 3 corresponding to the human mtDNA genome, the method comprising:
a) contacting the biological sample with a pair of PCR primers; and,
b) amplifying and quantifying the amount of the deletion using real-time PCR.

15. The method of claim 14, wherein one primer of the pair of primers has a nucleic acid sequence that specifically binds to a region of the deletion comprising a rejoining site after the deletion has recircularized.

16. The method of claim 14, wherein the deletion has the nucleotide sequence as set forth in SEQ ID NO: 1 or SEQ ID NO: 2.

17. A kit for:
(a) detecting breast or prostate cancer, or a genetic predisposition to breast or prostate cancer, in a human subject, the cancer being characterized by an elevated amount of mitochondrial DNA (mtDNA) having a deletion of 3926 base pairs within a region of mtDNA between nucleotides 12317 and 16254, with respect to SEQ ID NO. 3, of the human mtDNA genome;
(b) quantifying, in a biological sample from a human subject, the amount of mitochondrial DNA (mtDNA) having a deletion of 3926 base pairs within a region of mtDNA between nucleotides 12317 and 16254 of SEQ ID NO. 3 corresponding to the human mtDNA genome; and/or,
(c) quantifying in a biological sample from a human subject, the amount of a mtDNA deletion comprising 3926 base pairs from a region of mtDNA between nucleotides 12317 and 16254 of SEQ ID NO. 3 corresponding to the human mtDNA genome,
the kit comprising:
material for collecting or containing one or more biological samples; and,
at least one pair of PCR primers, wherein:
(i) one primer of the at least one pair of primers has a nucleic acid sequence that binds to a splice region of the mtDNA after removal of the deletion; and/or,
(ii) one primer of the at least one pair of primers has a nucleic acid sequence that binds to a region of the deletion comprising a rejoining site after the deletion has recircularized.

18. The kit of claim 17, wherein the one primer of the pair of primers has the nucleic acid sequence as set forth in SEQ ID NO: 4, 5, 6, or 10.

19. The kit of claim 18, wherein the pair of primers have the nucleic acid sequences of: (i) SEQ ID NOs: 4 and 5; (ii) SEQ ID NOs: 5 and 6; or (iii) SEQ ID NOs: 10 and 11.

20. The kit of claim 17, wherein the deletion has the nucleotide sequence as set forth in SEQ ID NO: 1 or SEQ ID NO: 2.


SEQUENCE LISTING (11 sequences)

SEQ ID NO: 1 - DNA, 3926 bp, Homo sapiens
taaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcatcttcctaattaccatactaatcttagttaccgctaacaacctattccaactgttcatcggctgagagggcgtaggaattatatccttcttgctcatcagttgatgatacgcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcaatactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaactaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagatgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaactgcaactccaaa

SEQ ID NO: 2 - DNA, 3926 bp, Homo sapiens
tgcaactccaaataaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcatcttcctaattaccatactaatcttagttaccgctaacaacctattccaactgttcatcggctgagagggcgtaggaattatatccttcttgctcatcagttgatgatacgcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcaatactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaactaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagatgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaac

SEQ ID NO: 3 - DNA, 16569 bp, Homo sapiens
  misc_feature (3107)..(3107): n is a, c, g, or t
gatcacaggtctatcaccctattaaccactcacgggagctctccatgcatttggtattttcgtctggggggtatgcacgcgatagcattgcgagacgctggagccggagcaccctatgtcgcagtatctgtctttgattcctgcctcatcctattatttatcgcacctacgttcaatattacaggcgaacatacttactaaagtgtgttaattaattaatgcttgtaggacataataataacaattgaatgtctgcacagccactttccacacagacatcataacaaaaaatttccaccaaaccccccctcccccgcttctggccacagcacttaaacacatctctgccaaaccccaaaaacaaagaaccctaacaccagcctaaccagatttcaaattttatcttttggcggtatgcacttttaacagtcaccccccaactaacacattattttcccctcccactcccatactactaatctcatcaatacaacccccgcccatcctacccagcacacacacaccgctgctaaccccataccccgaaccaaccaaaccccaaagacaccccccacagtttatgtagcttacctcctcaaagcaatacactgaaaatgtttagacgggctcacatcaccccataaacaaataggtttggtcctagcctttctattagctcttagtaagattacacatgcaagcatccccgttccagtgagttcaccctctaaatcaccacgatcaaaaggaacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactaagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaaccagagtgtagcttaacacaaagcacccaacttacacttaggagatttcaacttaacttgaccgctctgagctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctacnttcaaattcctccctgtacgaaaggacaagagaaataaggcctacttcacaaagcgccttcccccgtaaatgatatcatctcaacttagtattatacccacacccacccaagaacagggtttgttaagatggcagagcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaagaaatatgtctgataaaagagttactttgatagagtaaataataggagcttaaacccccttatttctaggactatgagaatcgaacccatccctgagaatccaaaattctccgtgccacctatcacaccccatcctaaagtaaggtcagctaaataagctatcgggcccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgtaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaaggaatcgaaccccccaaagctggtttcaagccaaccccatggcctccatgactttttcaaaaaggtattagaaaaaccatttcataactttgtcaaagttaaattataggctaaatcctatatatcttaatggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccctctagagcccactgtaaagctaacttagcattaaccttttaagttaaagattaagagaaccaacacctctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaattaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgaaccgaattggtatatagtttaaacaaaacgaatgatttcgactcattaaattatgataatcatatttaccaaatgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgtaaatatagtttaaccaaaacatcagattgtgaatctgacaacagaggcttacgaccccttatttaccgagaaagctcacaagaactgctaactcatgcccccatgtctaacaacatggctttctcaacttttaaaggataacagctatccattggtcttaggccccaaaaattttggtgcaactccaaataaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcatcttcctaattaccatactaatcttagttaccgctaacaacctattccaactgttcatcggctgagagggcgtaggaattatatccttcttgctcatcagttgatgatacgcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcaatactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaactaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagatgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaactgcaactccaaagccacccctcacccactaggataccaacaaacctacccacccttaacagtacatagtacataaagccatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggtcataaagcctaaatagcccacacgttccccttaaataagacatcacgatg

SEQ ID NO: 4 - DNA, 29 bp, Artificial
  Forward deletion detection primer
ttggtgcaactccaaagccacccctcacc

SEQ ID NO: 5 - DNA, 19 bp, artificial
  Reverse deletion detection primer.
aggatggtggtcaagggac

SEQ ID NO: 6 - DNA, 31 bp, Artificial
  Forward deletion detection primer
cccaaaaattttggtgcaactccaaagccac

SEQ ID NO: 7 - DNA, 12 bp, Homo sapiens
tgcaactccaaa

SEQ ID NO: 8 - DNA, 16 bp, Homo sapiens
ttggtgcaactccaaa

SEQ ID NO: 9 - DNA, 13 bp, Homo sapiens
gccacccctcacc

SEQ ID NO: 10 - DNA, 21 bp, Artificial Sequence
  Reverse primer
gaggggtggctttggagttgc

SEQ ID NO: 11 - DNA, 21 bp, Artificial Sequence
  Forward primer
aacagaggcttacgacccctt